(19) United States
(12) Patent Application Publication
(10) Pub. No.: US 2019/0236497 A1
(43) Pub. Date: Aug. 1, 2019

(54) SYSTEM AND METHOD FOR AUTOMATED MODEL SELECTION FOR KEY PERFORMANCE INDICATOR FORECASTING

(71) Applicant: KONINKLIJKE PHILIPS N.V., Eindhoven (NL)

(72) Inventors: Marcelo Santos, Lagoa Santa (BR); Jin Liu, Malden, MA (US); Eran Simhon, Boston, MA (US)

(21) Appl. No.: 16/253,454

(22) Filed: Jan. 22, 2019

Related U.S. Application Data
(60) Provisional application No. 62/783,268, filed on Dec. 21, 2018.
(60) Provisional application No. 62/624,427, filed on Jan. 31, 2018.

(51) Int. Cl. G06Q 10/04; G06Q 10/06; G06N 7/00; G16H 15/00; G16H 10/60

(52) U.S. Cl. CPC G06Q 10/04; G06Q 10/06393; G16H 10/60; G16H 15/00; G06N 7/00

(57) ABSTRACT

A method for KPI forecasting, comprising: (i) receiving an identification of one or more KPI to be forecast and a forecast horizon; (ii) extracting data received from a database for KPI forecasting; (iii) aggregating the extracted data; (iv) optionally removing one or more outliers from the aggregated data by identifying one or more possible outliers, presenting the outliers to a user, receiving information from the user comprising an identification of outliers, and removing the outliers; (v) fitting training data to a plurality of forecasting models; (vi) identifying a best fit forecasting model using test data; (vii) forecasting, using the best fit model, to generate KPI forecast data; (viii) evaluating the KPI forecast data for accuracy; and (ix) presenting the generated KPI forecast data to the user via a user interface.

### CROSS-REFERENCE TO RELATED APPLICATIONS

This application claims priority to both U.S. Provisional Patent Application Ser. No. 62/783,268, filed Dec. 21, 2018 and U.S. Provisional Application Ser. No. 62/624,427, filed on Jan. 31, 2018, the entire contents of which are incorporated herein by reference.

### FIELD OF THE DISCLOSURE

The following relates to systems and method for automated model selection for key performance indicator forecasting. By way of example, the following may describe forecasting of medical facility key performance indicators. However, it will become apparent that other key performance indicator forecasting may be performed using the methods and systems described herein.

### BACKGROUND

Forecasting of key performance indicators (KPIs) may be an important knowledge asset for a business and/or other facility. For example, KPI forecasting for hospital management teams is of increasing interest, particularly for clinical, operational, and financial KPIs. However, few businesses have a properly skilled team to create a KPI dashboard due to the complexity of the process.

For example, time series analysis and multiple linear regression are two major types of methods used for KPI forecasting, particularly for medical facility KPI forecasting. Current methods and systems require manually generating forecasting models or manually performing model fitting such as a time series analysis. This process not only involves a number of steps, such as checking autocorrelation function (ACF) and partial autocorrelation function (PACP) graphs before and after fitting several candidate models, but also requires subjective input from a skilled professional to determine a best fit model.

Despite various forecasting models developed over the years, different businesses and KPIs could have very different evolving patterns and thus require different models for forecasting. However, manual model fitting is time-consuming and requires specific statistical knowledge, which may not be available in a particular business environment, such as a hospital.

There are many challenges for a fully automatic forecasting pipeline. For example, data preparation requires determining how to identify and remove outliers without impacting a pattern. Data preparation also requires determining how to identify time series behavioral changes, such as those caused by internal policy changes (e.g., internal hospital policy changes), and eliminating the impact of those changes on forecasting. Fully automated forecasting pipelines may also require determining which method to use for model fitting and determining how to evaluate the model forecasting performance in a global and unbiased way. These and other drawbacks exist.

### SUMMARY OF THE DISCLOSURE

There is a continued need for methods and systems that perform key performance indicator (KPI) forecasting using automated model selection. Various embodiments and implementations herein are directed to a method and system configured to perform (KPI) forecasting. A user provides an indication of one or more KPI to be forecast together with a forecast horizon for the forecast. The system uses the received user information to extract electronic medical record (EMR) data received from an EMR database and aggregate the information into aggregated EMR data. Outliers in the aggregated data can optionally be removed by presenting at least a portion of the aggregated EMR data to a user, receiving an indication of one or more outliers in the aggregated EMR data, and removing based on the received indication one or more outliers in the aggregated EMR data. The system automatically fits training data to a plurality of forecasting models, where the training data comprises a portion of the aggregated data, and identifies a best fit forecasting model using test data, the test data comprising a portion of the aggregated data. The training data may comprise, for example, a remote portion of the aggregated data, and test data may comprise, for example, a recent portion of the aggregated data. The system the forecasts, using the identified best fit forecasting model and the aggregated data, the identified KPI(s) to generate KPI forecast data. The KPI forecast data is evaluated for accuracy over the identified forecast horizon using a forecast performance analysis using a predetermined threshold. If the KPI forecast data is determined not to be sufficiently accurate then one or more parameters of the best fit forecasting model is adjusted and KPI forecast data is generated using the modified best fit forecasting model. The system provides a report of the generated KPI forecast data to the user via a user interface, which may optionally include an indication to the user that the generated KPI forecast data comprises data quality or poor forecast performance below a predetermined threshold or quality level.

Generally, in one aspect, a method for key performance indicator (KPI) forecasting is provided. The method includes: (i) receiving an identification of one or more KPI to be forecast and a forecast horizon for the one or more identified KPI; (ii) extracting, based on the identified one or more KPI, data received from a database for KPI forecasting; (iii) aggregating, based on the identified forecast horizon, the extracted data; (iv) optionally removing one or more outliers from the aggregated data, comprising: identifying one or more possible outliers in the aggregated data, presenting the identified one or more possible outliers to a user via a user interface, receiving information from the user comprising an identification of one or more outliers in the identified one or more possible outliers, and removing, based on the identification received from the user, one or more of the possible outliers from the aggregated data; (v) automatically fitting training data to a plurality of forecasting models, the training data comprising a portion of the aggregated data; (vi) identifying a best fit forecasting model using test data, the test data comprising a portion of the aggregated data; (vii) forecasting, using the identified best fit forecasting model and the aggregated data, the one or more identified KPIs to generate KPI forecast data; (viii) evaluating the KPI forecast data for accuracy over the identified forecast horizon using a forecast performance analysis to determine, based on a predetermined threshold, that the KPI forecast data is sufficiently accurate over the identified forecast horizon or determining that the KPI forecast data is not sufficiently accurate over the identified forecast horizon; and (ix) presenting the generated KPI forecast data, and optionally forecast performance, to the user via a user interface.

According to an embodiment, the training data comprises a remote portion of the aggregated data, and test data comprises a recent portion of the aggregated data.

According to an embodiment, the method further includes the step of adjusting a parameter of the best fit forecasting model if the KPI forecast data is determined not to be sufficiently accurate, and generating KPI forecast data using the modified best fit forecasting model.

According to an embodiment, the aggregated data is presented via the user interface to the user as a line plot in real-time.

According to an embodiment, the method further includes the steps of: (i) analyzing the aggregated data to identify an anomaly in the data, the identification comprising a time period for the anomaly; and (ii) modifying the aggregated data to remove or minimize the identified anomaly.

According to an embodiment, the method further includes the step of deflating the extracted data using a consumer price index, if the identified one or more KPI is affected by the consumer price index.

According to an embodiment, optionally removing one or more outliers from the aggregated data further comprises the step of calculating an outlier possibility score for one or more of the possible outliers.

According to an embodiment, identifying a best fit model using the test data comprises evaluating an out-of-sample (test set) error for the aggregated data utilizing one or more models fitted using the training data.

According to an embodiment, the generated KPI forecast data is evaluated for accuracy using a Mean Absolute Scaled Error (MASE) analysis.

According to an embodiment, the step of providing an indication to the user that the generated KPI forecast data comprises data quality or poor forecast performance below a predetermined threshold or quality level.

According to an embodiment, the data is electronic medical record (EMR) data received from an EMR database.

According to an aspect is a system for key performance indicator (KPI) forecasting. The system includes: a user interface configured to receive an identification of one or more KPI to be forecast and a forecast horizon for the one or more identified KPI; a database comprising data for KPI forecasting; and a processor configured to: (i) extract, based on the identified one or more KPI, data from the database; (ii) aggregate, based on the identified forecast horizon, the extracted data; (iii) automatically fit training data to a plurality of forecasting models, the training data comprising a portion of the aggregated data; (iv) identify a best fit forecasting model using test data, the test data comprising a portion of the aggregated data; (v) forecast, using the identified best fit forecasting model and the aggregated data, the one or more identified KPIs to generate KPI forecast data; (vi) evaluate the KPI forecast data for accuracy over the identified forecast horizon using a forecast performance analysis to determine, based on a predetermined threshold, that the KPI forecast data is sufficiently accurate over the identified forecast horizon or determining that the KPI forecast data is not sufficiently accurate over the identified forecast horizon; and (vii) present the generated KPI forecast data, and optionally a forecast performance, to the user via the user interface.

According to an embodiment, the training data comprises a remote portion of the aggregated data, and test data comprises a recent portion of the aggregated data.

According to an embodiment, the processor is further configured to remove one or more outliers from the aggregated data, comprising: identify one or more possible outliers in the aggregated data; present the identified one or more possible outliers to a user via a user interface; receive information from the user comprising an identification of one or more outliers in the identified one or more possible outliers; and remove, based on the identification received from the user, one or more of the possible outliers from the aggregated data.

According to an embodiment, the processor is further configured to adjust a parameter of the best fit forecasting model if the KPI forecast data is determined not to be sufficiently accurate, and generating KPI forecast data using the modified best fit forecasting model.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for key performance indicator forecasting, in accordance with an embodiment.

FIG. 2 is a flowchart of a method for key performance indicator forecasting, in accordance with an embodiment.

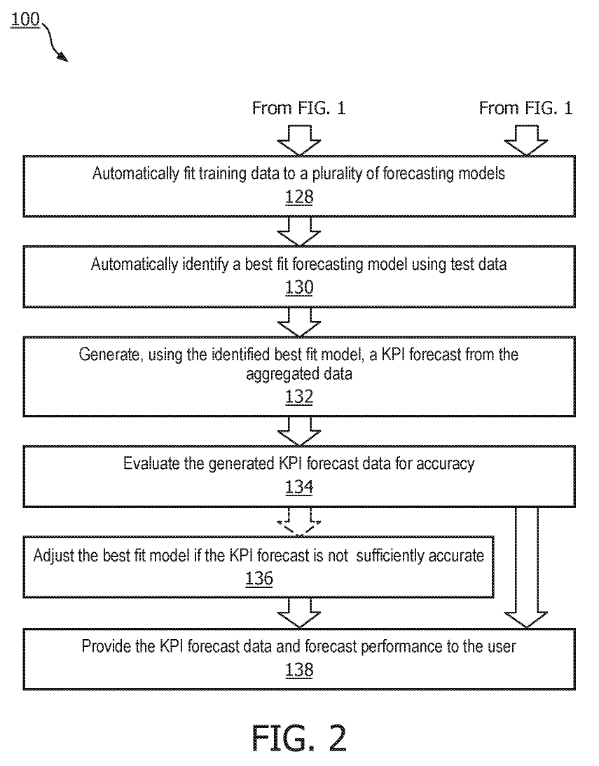

FIG. 3 is a schematic representation of a system for key performance indicator forecasting, in accordance with an embodiment.

FIG. 4 is a schematic representation of a system for key performance indicator forecasting, in accordance with an embodiment.

### DETAILED DESCRIPTION OF EMBODIMENTS

The present disclosure describes various embodiments of a system and method configured to perform key performance indicator (KPI) forecasting using automated model selection. More generally, Applicant has recognized and appreciated that it would be beneficial to provide a system that more accurately and more efficiently forecasts KPIs, which provides innumerable benefits. The system receives an indication of one or more KPI to be forecast together with a forecast horizon for the forecast, and automatically uses the received information to extract and aggregate electronic medical record data received from an EMR database. Outliers in the aggregated data can optionally be removed from the aggregated EMR data by the user. The system automatically fits training data to a plurality of forecasting models, where the training data comprising a portion of the aggregated data, and identifies a best fit forecasting model using the test data. The system the forecasts, using the identified best fit forecasting model and the aggregated data, the identified KPI(s) to generate KPI forecast data. The KPI forecast data is evaluated for accuracy over the identified forecast horizon using a forecast performance analysis using a predetermined threshold, and if the KPI forecast data is determined not to be sufficiently accurate then one or more parameters of the best fit forecasting model is adjusted and KPI forecast data is generated using the modified best fit forecasting model. The system provides a report of the generated KPI forecast data, and optionally a forecast performance, to the user via a user interface.

Automated model selection for improved KPI forecasting provides numerous benefits over manual KPI forecasting as well as KPI forecasting using predetermined or preselected forecasting models. For example, many professionals that utilize KPI forecasting in their industry are not qualified to select or modify a KPI forecasting model. Additionally, a forecasting model that works well for one location or industry may not work well for other locations or industries due to differences in the industries, management, customer/client demographics, location, and many other variables. For example, a forecasting model that accurately forecasts KPIs for one hospital may not accurately forecast KPIs for a second hospital. Additionally, a forecasting model that accurately forecasts KPIs for a location at one time of year may not accurately forecast KPIs at a different time of year. Accordingly, a system that can automatically select the best forecasting model saves time and energy not in the selection of a proper model, but in the potential negative impact of an inaccurate KPI forecast.

The systems and methods disclosed herein may be utilized in any industry utilizing KPIs and that may or does benefit from KPI forecasting. For example, the system may be implemented in a hospital setting that forecasts one or more KPIs, including but not limited to time periods such as daily, weekly, monthly, and many other. In addition to hospital settings, there are many other settings in which the system may be implemented.

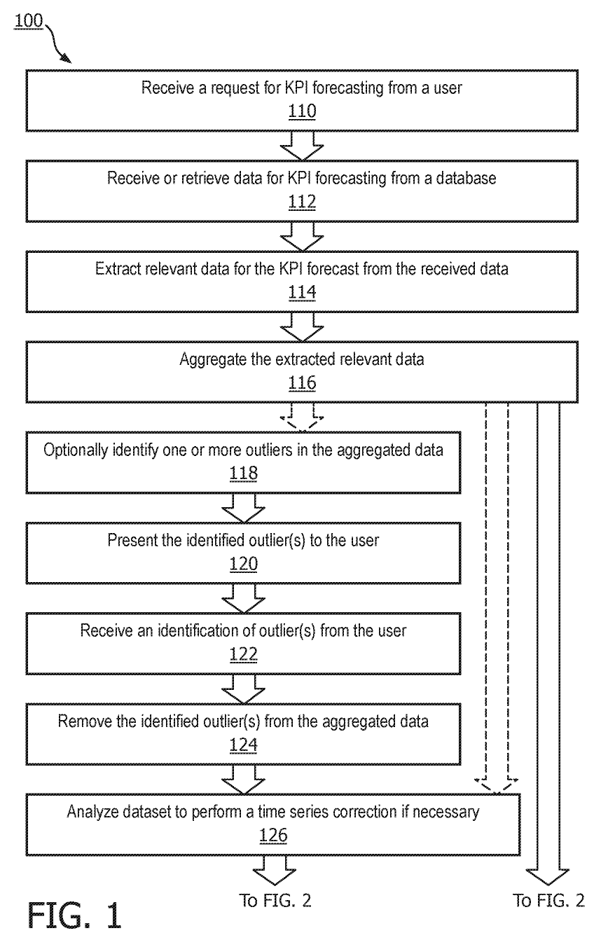

Referring to FIG. 1, in one embodiment, is a flowchart of a method 100 for key performance indicator (KPI) forecasting using automated model selection. The methods described in connection with the figures are provided as examples only, and shall be understood not to limit the scope of the disclosure. A forecasting system implementing this method can be any of the systems described or otherwise envisioned herein.

An automatic model selection algorithm may include data preparation automation, model fitting or selection automation, and forecasting as described or otherwise envisioned herein. Each of these functions may be executed on a system such as the systems described herein. And, each of these functions may be completed without the subjective input required by current models, thereby providing a technical improvement over existing modeling techniques.

By way of example, data preparation algorithms that automate the data preparation and remove subjective input may include, for example, data extraction and aggregation. Data extraction and aggregation may be performed in real-time, daily, weekly, monthly, and/or quarterly, for example. In the field of medical facilities, data that may be aggregated may include data from, for example, electronic medical records (EMRs) and/or electronic health records (EHRs). EMRs and/or EHRs may include, for example, patient data (e.g., patient identifier data, height, weight, Body Mass Index (BMI), average weight, average BMI, patient demographic data, and/or the like); administrative data (e.g., billing data, CPT code data, and/or the like); medical data (e.g., patient vital sign data, medical history data, diagnosis data, medication data, immunization data, allergy data, lab test data, lab results data, medical imaging data, at-home ambient sensor data, and/or the like); and/or workflow data (e.g., time between call date and appointment date, time between lab test and lab result, time between appointment and follow-up, and/or the like). Data preparation may also include data deflation based on whether a KPI is directly affected by a consumer price index stored in the system and updated in real-time and/or in batch processing. Data preparation may include automatic outlier detection. Automatic outlier detection may include analyzing a data distribution, dependency of neighboring records, and/or seasonality patterns. By way of example, an outlier possibility score may be calculated for each data point by adding abnormality of data distribution and dependency of neighboring records after removing a seasonal pattern. Data preparation may include automatically recognizing a time series pattern or behavior change and eliminating a time series pattern or behavior where required. By way of example, statistical or machine learning techniques may be employed to detect when different patterns and/or behaviors are present and when the pattern and/or behavior began. After identifying a change in a pattern and/or behavior, a corrective action may be performed on the dataset to account for this change. Data preparation may also include data transformation (e.g., differencing and/or log or power transformation) performed on the prepared data to achieve a stationary time series.

At step 110 of the method, an automated KPI forecasting system receives information from a user comprising a request for or information about a KPI forecast. The request may include an identification of one or more KPI that the user is requesting a forecast for from the system, and may include an identification of a forecast horizon for one or more of the requested KPIs. For example, the user may select a KPI for forecasting and may select a time period for the forecast such as a day, week, month, year, and/or any other time period.

The information may be provided via a user interface of a forecasting system. For example, the user may select or request a KPI via a pull-down menu, button selection, text entry, audible instruction, and/or any other method of input, selection, or request. The time period for the forecast may be similarly input, selected or requested.

At step 112 of the method, the automated KPI forecasting system receives current and historical data from a database to be utilized for forecasting. The data can be any data utilized to inform or create a KPI forecast, and may therefore depend on the industry for which the targeted KPI will be used. As just one non-limiting example, the KPI may be generated in a hospital setting, and thus the current and/or historical data may be electronic medical record (EMR) data. The data may also be information about the hospital, suppliers, and/or any other data that will or could facilitate a KPI forecast. The database may be any database comprising the required or desired data, and the database may be local or remote. Accordingly, the system may comprise a wired and/or wireless communication network or connection in order to communicate with a database.

The current and/or historical data may be pushed from the database to the automated KPI forecasting system or may be retrieved from the database by the KPI forecasting system. For example, the KPI forecasting system may be programmed or configured or directed to retrieve data from the database to generate a KPI, such as in response to a user request for a KPI forecast. Accordingly, the automated KPI forecasting system may comprise a user interface via with the user may program forecasts or may request a forecast.

At step 114 of the method, the automated KPI forecasting system extracts relevant data from the received current and historical data, for the KPI forecast. For example, the system can process and extract data from the received data using any method for data analysis and extraction. According to an embodiment, the data extraction may optionally be dependent upon the requested KPI forecast. For example, the data extraction may identify data that will be utilized for generation of the requested one or more KPI forecasts.

At step 116 of the method, the automated KPI forecasting system aggregates the extracted data. The aggregation can be performed based on any parameter, including but not limited to time period. For example, the KPI forecasting system may aggregate the extracted data by a time period such as hourly, daily, weekly, monthly, quarterly, yearly, and/or any other time period. Many other methods of aggregating the extracted data are possible.

According to an embodiment, the aggregated data may be presented via the user interface to the user in a manner enabling review of the data by the user. For example, the aggregated data may be presented to the user via a line plot in real-time, although many other display methods are possible. A line plot enables efficient and easily interpreted analysis by a user, although many other methods are possible, including annotated displays and others.

According to an embodiment, the aggregation may depend at least in part on the KPI forecast horizon selected by the user. As an example, if the user requests a forecast horizon of one week for a selected KPI, the system may extract and/or aggregate data relevant to the selected KPI for a time period equal to or smaller than the selected forecast horizon.

During, before, or after steps 114 and 116 of the method, the automated KPI forecasting system may optionally modify one or more values in the extracted and/or aggregated data. For example, the system may optionally perform deflation of one or more values based on whether the selected or identified KPI is affected by the consumer price index. The system may therefore store, retrieve, or otherwise receive or use a current or recent consumer price index value for the analysis. For example, the KPI forecasting system may be in communication with a paid or free service that provides an up-to-date consumer price index value.

According to another embodiment, the automated KPI forecasting system may optionally modify one or more values in the extracted and/or aggregated data by analyzing the aggregated data to identify an anomaly in the data. The identification may comprise, for example, a time period for the anomaly and behavior change recognition and elimination. For example, the system may apply a statistical or machine learning method to detect when different patterns or behaviors are present within a data series, as well as to determine when the new or different pattern or behavior began. When an anomaly is identified, the system may optionally modify the aggregated data to remove or minimize the identified anomaly. As just one example, the system may identify a sudden loss of data for a period of time during a time series required for a selected KPI, where a department was closed or data wasn't being obtained or provided for a period of time. The system may then ignore this time period or splice the data to obviate the missing data.

The user can optionally remove one or more outliers from the aggregated data, as shown in steps 118-124 of the method of FIG. 1. The automated KPI forecasting system may proceed directly from step 116 to 126 of the method without removing outliers, or may enable removal of one or more outliers. Accordingly, if outliers are optionally removed, at step 118 of the method the automated KPI forecasting system analyzes the aggregated data to identify one or more possible outliers in the aggregated data. Outliers may be identified from the data using any method for outlier identification, including a statistical analysis, best fit analysis, and/or any other method.

According to an embodiment, the automated KPI forecasting system may automatically detect one or more outliers in the aggregated data by analyzing the data distribution, utilizing dependency of neighboring records, and considering or otherwise utilizing a seasonality pattern, among many other methods for outlier identification. According to an embodiment, the automated KPI forecasting system may calculate an outlier possibility score for one or more data points and/or time points. For example, the system may utilize abnormalities within a data distribution and/or dependency of neighboring records, such as before or after removing a seasonal pattern, to identify outliers and generate an outlier possibility score.

At step 120 of the method, the automated KPI forecasting system presents the identified one or more possible outliers to a user via a user interface. The outliers may be presented using any method enabling viewing or understanding the identified outliers, particularly in relationship to the remainder of the data from which the identifier was identified. For example, the aggregated data may be presented in a line plot or other method for display. Outliers may be clearly identifiable or visible based on the display.

At step 122 of the method, the automated KPI forecasting system receives input from the user regarding the data and one or more outliers. The user may select individual outliers or may select a threshold or range for the system to subsequently identify outliers, and the selection may be performed via a pull-down menu, button selection, text entry, audible instruction, and/or any other method of input, selection, or request. For example, the user may provide or select a threshold, range, or other variable or parameter that then identifies outliers within the aggregated data. Alternatively, the user may only select individual data to identify the data as outliers.

At step 124 of the method, the automated KPI forecasting system removes one or more outliers from the aggregated data based on the input received from the user via the user interface. For example, the system may use a threshold or range provided by the user to identify which of the identified possible outliers and/or other data are user-identified outliers, and will remove those outliers. As another example, the system may remove from the aggregated data any outliers directly or otherwise identified by the user.

At step 126 of the method, the KPI forecasting system optionally performs a time series analysis of the aggregated data for stationarity testing, and to optionally transform the aggregated data to address any identified issues in the stationarity testing. According to an embodiment, the KPI forecasting system analyzes the aggregated data to determine whether one or more series of data is stationary such that it does not have statistical properties that change with time. The analysis may comprise, for example, a KPSS test. After identifying any nonstationarity, the system can perform a data transformation such as differencing, log or power transformation, and/or any other method, to achieve a stationary time series that can be utilized for downstream analysis.

At step 128 of the method, the KPI forecasting system automatically fits training data to a plurality of forecasting models. According to an embodiment, the aggregated data is split into a training set (in sample) and a test set (out of sample), where the training data comprises a relatively remote portion of the aggregated data and the test data comprises a relatively recent portion of the aggregated data. For example, the automatic model selection may include using the data prepared during data preparation to automatically fit one or more models to the data. By way of example, model selection may automatically fit multiple models using time-series analysis models, multiple linear regression models, and/or neural network models, and/or a combination of those models using the training data set. Where data preparation detects a seasonality pattern, the seasonality models (e.g., seasonal autoregressive integrated moving average (ARIMA) models and/or TBATS models) may be fit. Where data preparation does not detect seasonality and/or seasonality is removed, non-seasonal models may be fit. Model selection may include determining a best fit model for each method type, for example, based on Akaike information criterion (AIC) and/or Bayesian information criterion (BIC) using the data from the training set.

The forecasting model may be any model utilized for forecasting, and many such models are available. For example, time-series analysis models, multiple linear regression models, and neural network models, among other models, may be utilized. Other types of possible models include seasonal models such as seasonal AutoRegressive Integrated Moving Average (ARIMA) or TBATS models, although only non-seasonal models may be used if there is no seasonality, or seasonality within the training data may be removed or ignored.

The forecasting model may be, for example, a multiple linear regression model with or without variable selection of parameters such as dates (is there a holiday or other date that will affect the KPI forecast?), patient information (are there demographics or patient information that will affect the KPI forecast?), social information (does the area the industry serves affect the KPI forecast?), and/or anything else that might affect the selected KPI.

The forecasting model may be, for example, a neural network model such as a feedforward or recurrent neural network model, among many other possibilities. According to yet another embodiment, the KPI forecasting system may utilize a combination of two or more of these or other models to analyze the data and produce a KPI forecast.

The KPI forecasting system will collect the KPI forecast data from each of the plurality of forecasting models used to analyze the training data. The system can use the data immediately or can store the data to use it as a later point or date.

At step 130 of the method, the automated KPI forecasting system automatically identifies one of the plurality of forecasting models as a best fit forecasting model using the test data. The system uses the collected KPI forecast data to perform a best fit analysis. Any method of identifying a best fit model may be utilized. For example, the system may evaluate out-of-sample (test set) error of all the identified best models using a method such as root-mean-square error (RMSE) analysis among others. For example, the system may use RMSE to measure the differences between the KPI forecast values predicted by each of the identified two or more best fit models and expected or observed KPI forecast values based on the test set. The best fit model that with KPI forecast values that most closely align with or match or are otherwise most similar to the expected or observed KPI forecast values may be selected as the single best fit model for subsequent analysis.

At step 132 of the method, the automated KPI forecasting system uses the identified best fit forecasting model from step 130 to forecast one or more of the KPIs requested by the user, thereby generating KPI forecast data. The identified best fit forecasting model from step 130 may of course be any of the plurality of forecasting models used to generate forecast data using the test data. The generated KPI forecast data may be utilized immediately and/or may be temporarily or permanently stored for subsequent downstream analysis.

At step 134 of the method, the automated KPI forecasting system evaluates the KPI forecast data for accuracy over the identified forecast horizon using a forecast performance analysis. Accordingly, the system will thus determine, based on a predetermined threshold, that the KPI forecast data is sufficiently accurate over the identified forecast horizon or determine that the KPI forecast data is not sufficiently accurate over the identified forecast horizon. Any model for evaluating KPI forecast data for accuracy may be used for the analysis.

According to an embodiment, the KPI forecasting system evaluates the KPI forecast data for accuracy using a mean absolute scaled error (MASE) analysis, although many other methods are possible. For example, the system may use a MASE analysis on out-of-sample or test set data to determine where a forecast is accurate considering the forecast horizon. The forecast error on out-of-sample or test set data can be compared to the forecast error on the aggregated data using the naive method. These results can be analyzed using certain criteria to evaluate the model for accuracy. For example, criteria for determining the accuracy of forecasting may include better than naive (i.e., random walk) adjusted by the forecast horizon. For example, after obtaining an increase slope of MASE with an increasing forecast horizon based on a previously published or determined MASE, a benchmark for MASE may be determined using, for example, the following formula:

Mean(Σₙ₌₁^{N}Σₕ₌₁ⁿMASE(h)) Eq. (1)

where MASE(h)=(h−1)*a+1, where h is a forecast horizon, a is an increasing rate. When the forecast horizon, h, equals 1 (only forecast for one step ahead), MASE=1 which means that the model performs the same as the naive method.

The naive (i.e., random walk) may assume, for example, that a KPI will be similar or identical to a current value or a historical value. For example, the random walk may assume that a hospital will have the same number of patients next week that it has this week, or tomorrow that it has today. Thus the naive MASE may simply be 1 when the forecast horizon is 1. As expected, possible and allowable error is expected increase as forecasted time increases. Accordingly, the MASE threshold may increase as time increases to allow for this possible and allowable error. This is just one example for evaluating the KPI forecast data for accuracy, and other methods are possible.

According to an embodiment, the accuracy analysis may demonstrate or suggest that the KPI forecast from the selected best fit model is better than or not better than a previous model for KPI forecast or the naive analysis, such as by comparing the KPI forecast or analyzing data to a predetermined threshold or other result. For example, the system may use a MASE benchmark as a threshold for determining accuracy.

According to an embodiment, if the accuracy analysis shows or suggests that the KPI forecast from the selected best fit model is not sufficiently accurate, for example that the KPI forecast from the selected best fit model is not different from or better than naive, then the KPI forecast is flagged or otherwise identified as inaccurate (or not sufficiently accurate). Similarly, if the accuracy analysis shows or suggests that the KPI forecast from the selected best fit model is sufficiently accurate, for example that the KPI forecast from the selected best fit model is different from or better than naive, then the KPI forecast is flagged or otherwise identified as accurate (or sufficiently accurate).

At optional step 136 of the method, the automated KPI forecasting system adjusts a parameter of the best fit forecasting model if the KPI forecast data is determined not to be sufficiently accurate, and generating KPI forecast data using the modified best fit forecasting model. For example, the automated KPI forecasting system may adjust one or more parameters of the identified best fit model used to analyze the aggregated data. The adjustment may be an automatic adjustment, or may be based at least in part on one or more of the values from step 134 of the method.

At step 138 of the method, the automated KPI forecasting system uses the selected best model and all aggregated data to generate KPI forecast data for the near future defined by the forecast horizon and presents the KPI forecast data, as well as forecast performance, to the user via a user interface. The generated KPI forecast data may be provided via any means of communication. According to an embodiment, the KPI forecast data may be presented via the user interface to the user in a manner enabling review of the data by the user. For example, the KPI forecast data may be presented to the user via a line plot in real-time, although many other display methods are possible. A line plot enables efficient and easily interpreted analysis by a user, although many other methods are possible, including annotated displays and others.

According to an embodiment, the KPI forecasting system presents the generated KPI forecast data to the user together with forecast performance (e.g., an alert or indication of suspect data quality or poor forecast performance below a predetermined threshold or quality level). This may be based on the analysis in step 134 of the method, among other possible methods of evaluating the quality of the data. For example, KPI forecast data that is similar to or worse than naive walk may be identified as having suspect data quality or poor forecast performance, while KPI forecast data that better than naive walk may be identified as being of high-quality forecast performance. The indication of data quality can be a ranking, sound, color, or any other indication of quality.

According to an embodiment, the KPI forecasting system may optionally transform some or all of the data before presentation via the user interface. For example, any data inverse transformation (e.g., differencing and/or log or power transformation) corresponding to the data transformation performed on the prepared data to achieve a stationary time series, among other possibilities.

Accordingly, in addition to the algorithms described herein that will be executed on systems, such as those described herein, the various embodiments described herein may include an interactive user interface that automatically provides KPI forecasting data. By way of example, an interactive user interface may include a display of original data as line plots in real-time for users to be informed of possible outliers, pattern and/or behavior changes and needs from transformation. An interactive user interface may include functionality that allows users to select an option for data preparation, such as particular KPI selection, sector selection, outlier removal criteria, and/or data transformation, as well as a forecasting horizon. An interactive user interface may display a forecasting result and accuracy. An interactive user interface and/or other output (e.g., audio, visual, and/or data transmission to user device such as a wearable device and/or smartphone) may include providing an alert when the modeling described herein determines that the data quality is poor and/or a forecast performance or evaluation indicates a poor forecast.

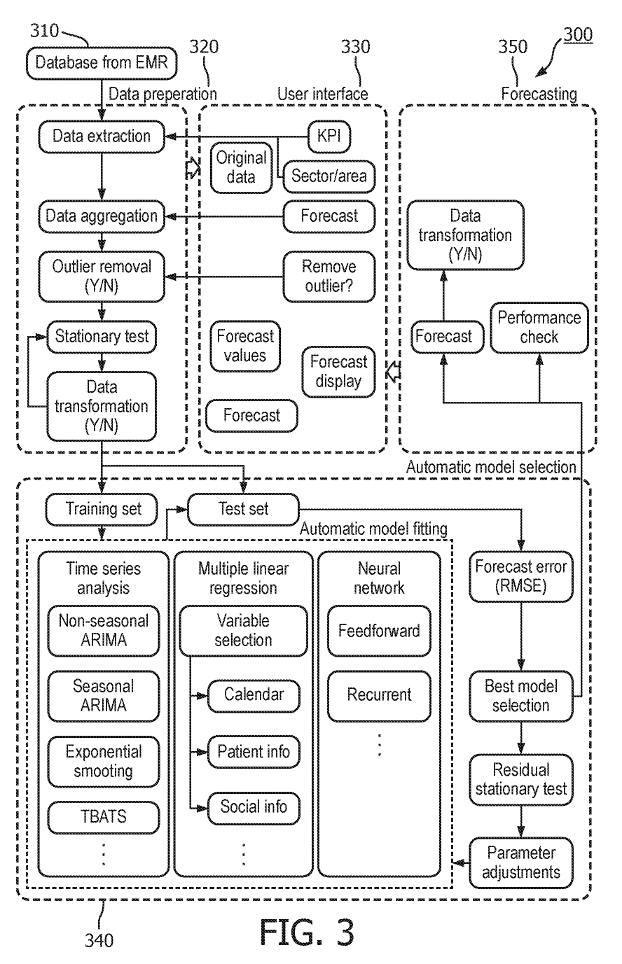

Referring to FIG. 3 is a schematic representation of an automated KPI forecasting system 300, in accordance with an embodiment. KPI forecasting system 300 receives information from a user comprising a request for or information about a KPI forecast. The request is typically provided via a user interface module or process 330. The request may include an identification of one or more KPI that the user is requesting a forecast for from the system, and may include an identification of a forecast horizon for one or more of the requested KPIs. For example, the user may select a KPI for forecasting and may select a time period for the forecast such as a day, week, month, year, and/or any other time period.

To perform the KPI forecast, the KPI forecasting system 300 comprises a data preparation module or process 320 which performs one or more steps or analyzes to generate aggregated data for KPI forecasting. For example, the automated KPI forecasting system receives current and historical data from a database 310 to be utilized for forecasting. The data can be any data utilized to inform or create a KPI forecast, and may therefore depend on the industry for which the targeted KPI will be used.

The data preparation module or process 320 can extract relevant data from the received current and historical data for the KPI forecast, and aggregate the extracted data. The aggregation can be performed based on any parameter, including but not limited to time period. The data preparation module or process 320 can facilitate the removal of outliers by presenting possible outliers to a user and receiving data from the user regarding an identification of outliers. The data preparation module or process 320 can also automatically remove or modify anomalous data or adjust data based on the consumer price index, among other possible modifications. The data preparation module or process 320 may optionally perform a time series analysis of the aggregated data for stationarity testing, and may optionally transform the aggregated data to make the time series stationary. Many other modifications and analyses are possible. The data preparation module or process 320 therefore generates aggregated data that can be used to generate a KPI forecast.

The KPI forecasting system 300 further comprises an automatic model selection module or process 330 that identifies a single best fit model that will be used to generate a KPI forecast using the aggregated data. There are many possible methods for identifying a single best fit model. According to an embodiment, the automatic model selection module or process 330 the KPI forecasting system automatically fits training data to a plurality of forecasting models using the training set data. Any forecasting model may be utilized to analyze the data. The KPI forecasts from the plurality of methods are collected and analyzed to identify one best fit model for each method. Any method of identifying a best fit model for each method may be utilized with the training data. For example, the KPI forecasting system may use Akaike information criterion (AIC) and/or Bayesian information criterion (BIC) using the training set data to identify a best fit model. According to an embodiment, if there are two or more best fit models identified from two or more methods, the KPI forecasting system automatically analyzes the two or more best fit models to identify a single best model type for forecasting the KPI using the test data. Any method of selecting a single best model from among a plurality of possible best fit models may be used. For example, the system may evaluate out-of-sample (test set) error of all the identified best models using a method such as root-mean-square error (RMSE) analysis among others.

The KPI forecasting system uses the identified best fit forecasting model to forecast one or more of the KPIs requested by the user, thereby generating KPI forecast data. The generated KPI forecast data may be utilized immediately and/or may be temporarily or permanently stored for subsequent downstream analysis. The KPI forecasting system evaluates the KPI forecast data for accuracy over the identified forecast horizon using a forecast performance analysis on the test data. According to an embodiment, the KPI forecasting system evaluates the KPI forecast data for accuracy using a mean absolute scaled error (MASE) analysis, although many other methods are possible. The KPI forecasting system may adjust a parameter of the best fit forecasting model if the KPI forecast data is determined not to be sufficiently accurate, and may generate KPI forecast data using the modified best fit forecasting model.

KPI forecasting system 300 further comprises a forecasting module or process 340 to generate final KPI forecasts using the identified best fit forecasting model. Once the KPI forecasts are generated, the KPI forecasting system presents the generated KPI forecast data to the user via the user interface module or process 220. The KPI forecast data may be presented via the user interface to the user in a manner enabling review of the data by the user. For example, the KPI forecast data may be presented to the user via a line plot in real-time, although many other display methods are possible. The KPI forecast data may be presented to the user together with an alert or indication of suspect data quality or poor forecast performance below a predetermined threshold or quality level.

According to an embodiment, a report may be a visual display, a printed text, an email, an audible report, a transmission, and/or any other method of conveying information. The report may be provided locally or remotely, and thus the system or user interface may comprise or otherwise be connected to a communications system. For example, the system may communicate a report over a communications system such as the internet or other network. May other methods of providing, recording, reporting, or otherwise making the KPI forecasts available are possible.

According to an embodiment, the methods and systems described or otherwise envisioned herein may further comprise utilizing, by a decision maker, the KPI forecast to make a forecast decision. Once the user receives the KPI forecast, the decision maker (such as an administrator, purchaser, budgeter, or any other decision maker that uses KPI forecast information either directly or indirectly) may review and utilize that forecast information to make a decision about the subject matter of the KPI. For example, if the KPI forecast indicates that a hospital may have higher occupancy in the coming week, the decision maker may implement measures or decisions that enable, facilitate, or maximize that higher occupancy.

Decision-making regarding the provided KPI forecast may optionally involve the forecast performance information. For example, a decision maker may rely more upon a KPI forecast with an indication of a higher forecast performance and may rely less upon a KPI forecast with an indication of a lower forecast performance. Thus, a decision maker may utilize fewer or additional sources of information in addition to the KPI forecast, based on the forecast performance, to make a decision.

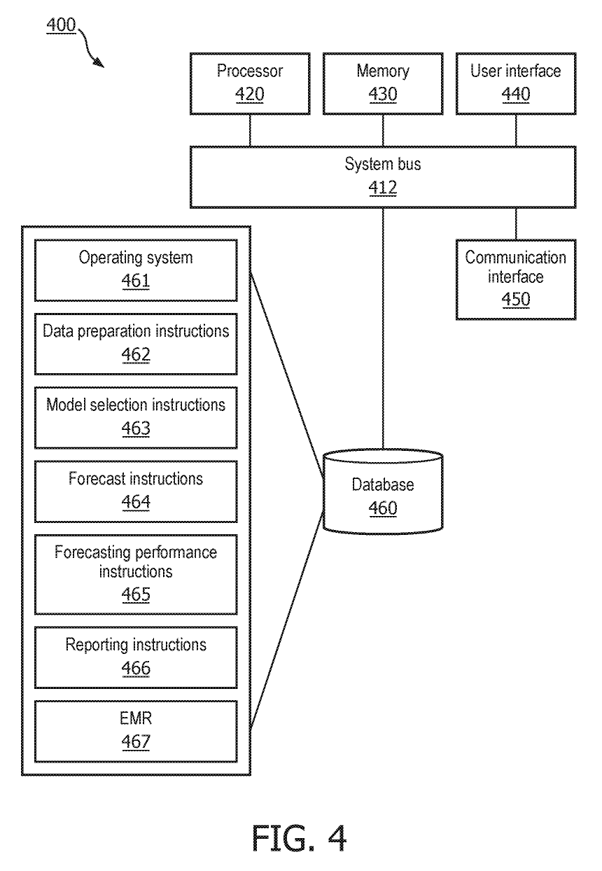

Referring to FIG. 4, in one embodiment, is one embodiment, is a schematic representation of an automated KPI forecasting system 400 configured to generate one or more KPI forecasts utilizing a best fit forecasting model. System 400 may be any of the systems described or otherwise envisioned herein, and may comprise any of the components described or otherwise envisioned herein.

According to an embodiment, system 400 comprises one or more of a processor 420, memory 430, user interface 440, communications interface 450, and storage 460, interconnected via one or more system buses 412. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 430 or storage 460 or otherwise processing data to, for example, perform one or more steps of the method. Processor 420 may be formed of one or multiple modules. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 430 can take any suitable form, including a non-volatile memory and/or RAM. The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user. The user interface can be any device or system that allows information to be conveyed and/or received, and may include a display, a mouse, and/or a keyboard for receiving user commands. In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (NIC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

Database or storage 460 may include one or more machine-readable storage media such as read-only memory (ROM), random-access memory (RAM), magnetic disk storage media, optical storage media, flash-memory devices, or similar storage media. In various embodiments, database 460 may store instructions for execution by processor 420 or data upon which processor 420 may operate. For example, database 460 may store an operating system 461 for controlling various operations of system 400. Database 460 may also store electronic medical records (EMR) 467, which may be electronic medical records or any other data necessary for the KPI forecast, such as data specific to the industry for which the KPI will be generated.

It will be apparent that various information described as stored in database 460 may be additionally or alternatively stored in memory 430. In this respect, memory 430 may also be considered to constitute a storage device and database 460 may be considered a memory. Various other arrangements will be apparent. Further, memory 430 and database 460 may both be considered to be non-transitory machine-readable media. As used herein, the term non-transitory will be understood to exclude transitory signals but to include all forms of storage, including both volatile and non-volatile memories.

While KPI forecasting system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where one or more components of system 400 is implemented in a cloud computing system, the various hardware components may belong to separate physical systems. For example, processor 420 may include a first processor in a first server and a second processor in a second server. Many other variations and configurations are possible.

According to an embodiment, KPI forecasting system 400 may store or comprise one or more algorithms, engines, and/or instructions to carry out one or more functions or steps of the methods described or otherwise envisioned herein. For example, database 460 may store electronic medical records and/or electronic health records, or other data used to generate KPIs. The system may comprise, among other instructions, data preparation instructions 462, model selection instructions 463, forecasting instructions 464, forecasting performance instructions 465, and/or reporting instructions 466. The system may store additional software components required to execute the functionality described herein, which also may control operations of hardware 400.

According to an embodiment, data preparation instructions 462 direct the system to request or receive current and/or historical data from a local and/or remote database to be utilized for forecasting. The data can be any data utilized to inform or create a KPI forecast, and may therefore depend on the industry for which the targeted KPI will be used. A request for a KPI forecast may typically be received via user interface 440 or may be automated to be generated periodically or in response to a trigger. The instructions direct the system to extract relevant data from the received current and historical data for the KPI forecast, and aggregate the extracted data. The aggregation can be performed based on any parameter, including but not limited to time period. The data preparation instructions 462 can facilitate the removal of outliers by presenting possible outliers to a user and receiving data from the user regarding an identification of outliers. The data preparation instructions 462 can also automatically remove or modify anomalous data or adjust data based on the consumer price index, among other possible modifications. The data preparation instructions 462 may optionally perform a time series analysis of the aggregated data for stationarity testing, and may optionally transform the aggregated data to address any identified issues in the stationarity testing. Many other modifications and analyses are possible. The data preparation instructions 462 therefore generate aggregated data that can be used to generate a KPI forecast.

According to an embodiment, model selection instructions 463 direct the system to identify a single best fit model that will be used to generate a KPI forecast using the aggregated data. There are many possible methods for identifying a single best fit model. According to an embodiment, the model selection instructions 463 direct the KPI forecasting system to fit training data to a plurality of forecasting models using the training set data. The KPI forecasts from the plurality of methods are collected and analyzed to identify one best fit model for each method. Any method of identifying a best fit model for each method may be utilized with the training data. For example, the KPI forecasting system may use Akaike information criterion (AIC) and/or Bayesian information criterion (BIC) using the training set data to identify a best fit model. According to an embodiment, if there are two or more best fit models identified from two or more methods, the KPI forecasting system automatically analyzes the two or more best fit models to identify a single best model type for forecasting the KPI using the test data. Any method of selecting a single best model from among a plurality of possible best fit models may be used. For example, the system may evaluate out-of-sample (test set) error of all the identified best models using a method such as root-mean-square error (RMSE) analysis among others.

According to an embodiment, forecasting instructions 464 direct the system to use the identified best fit forecasting model to forecast one or more of the KPIs requested by the user, thereby generating KPI forecast data. The generated KPI forecast data may be utilized immediately and/or may be temporarily or permanently stored for subsequent downstream analysis.

According to an embodiment, forecasting performance instructions 465 direct the system to evaluate the KPI forecast data for accuracy over the identified forecast horizon using a forecast performance analysis. According to an embodiment, the KPI forecasting system evaluates the KPI forecast data for accuracy using a mean absolute scaled error (MASE) analysis, although many other methods are possible. The KPI forecasting system may adjust a parameter of the best fit forecasting model if the KPI forecast data is determined not to be sufficiently accurate, and may generate KPI forecast data using the modified best fit forecasting model.

According to an embodiment, reporting instructions 466 direct the system to generate, report, and/or provide the generated KPI forecast(s) to the user via the user interface 440. This could be created in memory or a database, displayed on a screen or other user interface or otherwise provided. The KPI forecast data may be presented to the user together with an alert or indication of suspect data quality or poor forecast performance below a predetermined threshold or quality level. A report may be a visual display, a printed text, an email, an audible report, a transmission, and/or any other method of conveying information. The report may be provided locally or remotely, and thus the system or user interface may comprise or otherwise be connected to a communications system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A computer-implemented method for key performance indicator (KPI) forecasting, comprising:
receiving (i) an identification of one or more KPI to be forecast; and (ii) a forecast horizon for the one or more identified KPI;
extracting, based on the identified one or more KPI, data received from a database for KPI forecasting;
aggregating, based on the identified forecast horizon, the extracted data;
optionally removing one or more outliers from the aggregated data, comprising: (i) identifying one or more possible outliers in the aggregated data; (ii) presenting the identified one or more possible outliers to a user via a user interface; (iii) receiving information from the user comprising an identification of one or more outliers in the identified one or more possible outliers; and (iv) removing, based on the identification received from the user, one or more of the possible outliers from the aggregated data;
automatically fitting training data to a plurality of forecasting models, the training data comprising a portion of the aggregated data;
identifying a best fit forecasting model using test data, the test data comprising a portion of the aggregated data;
forecasting, using the identified best fit forecasting model and the aggregated data, the one or more identified KPIs to generate KPI forecast data;
evaluating the KPI forecast data for accuracy over the identified forecast horizon using a forecast performance analysis to determine, based on a predetermined threshold, that the KPI forecast data is sufficiently accurate over the identified forecast horizon or determining that the KPI forecast data is not sufficiently accurate over the identified forecast horizon; and
presenting the generated KPI forecast data to the user via a user interface.

2. The method of claim 1, further comprising the step of adjusting a parameter of the best fit forecasting model if the KPI forecast data is determined not to be sufficiently accurate, and generating KPI forecast data using the modified best fit forecasting model.

3. The method of claim 1, wherein the aggregated data is presented via the user interface to the user as a line plot in real-time.

4. The method of claim 1, further comprising the steps of: (i) analyzing the aggregated data to identify an anomaly in the data, the identification comprising a time period for the anomaly; and (ii) modifying the aggregated data to remove or minimize the identified anomaly.

5. The method of claim 1, further comprising the step of deflating the extracted data using a consumer price index, if the identified one or more KPI is affected by the consumer price index.

6. The method of claim 1, wherein optionally removing one or more outliers from the aggregated data further comprises the step of calculating an outlier possibility score for one or more of the possible outliers.

7. The method of claim 1, wherein identifying a best fit model comprises evaluating an out-of-sample (test set) error for the aggregated data utilizing one or more models fitted using the training data.

8. The method of claim 1, wherein the generated KPI forecast data is evaluated for accuracy using a Mean Absolute Scaled Error (MASE) analysis.

9. The method of claim 1, further comprising the step of providing an indication to the user that the generated KPI forecast data comprises data quality or poor forecast performance below a predetermined threshold or quality level.

10. The method of claim 1, wherein the data is electronic medical record (EMR) data received from an EMR database.

11. A system for key performance indicator (KPI) forecasting, comprising:
a user interface configured to receive: (i) an identification of one or more KPI to be forecast; and (ii) a forecast horizon for the one or more identified KPI;
a database comprising data for KPI forecasting; and
a processor configured to: (i) extract, based on the identified one or more KPI, data from the database; (ii) aggregate, based on the identified forecast horizon, the extracted data; (iii) automatically fit training data to a plurality of forecasting models, the training data comprising a portion of the aggregated data; (iv) identify a best fit forecasting model using test data, the test data comprising a portion of the aggregated data; (v) forecast, using the identified best fit forecasting model and the aggregated data, the one or more identified KPIs to generate KPI forecast data; (vi) evaluate the KPI forecast data for accuracy over the identified forecast horizon using a forecast performance analysis to determine, based on a predetermined threshold, that the KPI forecast data is sufficiently accurate over the identified forecast horizon or determining that the KPI forecast data is not sufficiently accurate over the identified forecast horizon; and (vii) present the generated KPI forecast data to the user via the user interface.

12. The system of claim 11, wherein the data is electronic medical record (EMR) data and the database is an EMR database.

13. The system of claim 11, wherein the processor is further configured to remove one or more outliers from the aggregated data, comprising:
identify one or more possible outliers in the aggregated data;
present the identified one or more possible outliers to a user via a user interface;
receive information from the user comprising an identification of one or more outliers in the identified one or more possible outliers; and
remove, based on the identification received from the user, one or more of the possible outliers from the aggregated data.

14. The system of claim 11, wherein the processor is further configured to adjust a parameter of the best fit forecasting model if the KPI forecast data is determined not to be sufficiently accurate, and generating KPI forecast data using the modified best fit forecasting model.

15. The system of claim 11, wherein identifying a best fit model comprises evaluating an out-of-sample (test set) error for the aggregated data utilizing one or more models fitted using the training data.
